(19) United States
(12) Patent Application Publication
(10) Pub. No.: US 2003/0203959 A1
(43) Pub. Date: Oct. 30, 2003

(54) Novel modulators of potassium channels

(75) Inventors: Heiko Rauer, Muenchen (DE); Wael Saeb, Martinsried (DE); Gabriel Garcia, Muenchen (DE); Bernd Kramer, Aachen (DE); Juergen Kraus, Grafenrheinfeld (DE); Claudia Klemenz, Gilching (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: 4SC AG, Martinsried (DE)

(21) Appl. No.: 10/376,264

(22) Filed: Mar. 3, 2003

Related U.S. Application Data
(60) Provisional application No. 60/361,299, filed on Mar. 4, 2002.

(51) Int. Cl. A61K 31/405; C 07D 4 3/02

(52) U.S. Cl. 514/414; 548/465

(57) ABSTRACT

The invention relates to the use of a compound of the Formula (I)

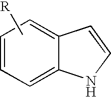

or a salt, a physiologically functional derivative, or a prodrug thereof as a medicament, wherein

R is a monocyclic or polycyclic substituted or unsubstituted aromatic ring system which may contain one or more groups X and which contains at least one aromatic ring;

X is selected from the group consisting of S, O, N, NR′, SO or SO₂;

R is optionally substituted by one to four substituents which are independently selected from the group consisting of halogen, CF₃, OCF₃, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, alkylaryl, alkylsulfonyl, H, hydroxy, aryl, heteroaryl, —NR′OR′, CN, alkylsulfinyl, arylsulfonyl, heteroarylsulfonyl, SO₃R′, NO₂, —CO—NR′R¹, arylalkyl-O—, —O-aryl, —O-heteroaryl, arylalkyl-S—, —S-aryl, —S-heteroaryl, —NR¹—SO₂R′, —SO₂—NR^{I}-alkyl, —SO₂—NR¹-aryl, and —SO₂—N¹-heteroaryl.

The present invention relates to potassium channel modulating indole derivatives. These compounds are useful in the treatment or alleviation of disorders and conditions associated with, or dependent on the membrane potential or conductance of cells in mammals, including a human. The present method also provides a method for the manufacture of medicaments and pharmaceutical compositions comprising the K⁺ channel modulating agents. The agents of the invention are useful for the treatment or alleviation of diseases, disorders, and conditions associated with or responsive to the modulation of potassium channels.

Potassium channels (K⁺ channels) are present in nearly all cells and play a crucial role in a wide variety of cellular regulation processes due to modulation of the membrane potential. K⁺ channels can be regulated by changes in membrane voltage, internal Ca²⁺ concentration, phosphorylation, and multiple other cellular mechanisms (Hille, B., Ionic channels in excitable membranes, 2^{nd} ed, Sinauer Assc. (1992)). The family of potassium channels can be divided into several subfamilies, one being the group of Ca²⁺-activated K⁺ channels. The potassium channel BK belongs to this subfamily of Ca²⁺-activated K⁺ channels (K_{Ca}) and shows a large single channel conductance of ˜150 pS. The BK channel (or MaxiK), encoded by the Slo gene, is mainly regulated by the internal Ca²⁺ concentration and membrane voltage as well as β-subunit modulation, phosphorylation states, and other cellular mechanisms (Nelson M. T. et al., Science 270, 633-637 (1995); Levitan, I. B., Annu. Rev. Physiol., 56, 193-212 (1994); Vergara et al., Curr. Opin. Neurobiol. 8, 321-329 (1998); McManus, O. B., Neuron, 14, 645-650 (1995)). Large conductance, Ca²⁺-activated BK channels are ubiquitously expressed, except in myocardial tissue, and play a key role, e.g. in smooth muscle tone, neuron firing, and cell secretion (Toro, L. et al., From ion channels to cell to cell conversations, Plenum Press, NY 47-65, (1997); Fox, A. J. et al., J. Clin. Invest., 99, 513-519 (1997); Nelson M. T. et al, Science 270, 633-637 (1995); Lingle C:J., et al, Ion channels, 4, 4, 261-301 (1996)). The opening of BK channels leads to a shift of the membrane potential towards the potassium reversal potential causing hyperpolarization of the cell. Due to its large single channel conductance the opening of only few BK channels can produce a significant leftward shift of the membrane potential due to the increased K⁺ conductance. Such mechanisms are important for example in smooth muscle cells, where hyperpolarization caused by BK channel opening leads to a relaxation and therefore a reduced vascular tone, or in neuronal tissue, where BK channel opening counteracts depolarisation and can limit the hyperactivating and/or damaging Ca²⁺ entry under different disease conditions. Inhibition of BK channels can maintain or lead to a more depolarized membrane potential of the cell and therefore maintain or prolong cellular processes depending on cellular depolarization.

Other members of the subfamily of Ca²⁺-activated K⁺ channels (K_{Ca}) are SK_{Ca} (SK_{Ca}-1,2,3) and IK_{Ca} channels, with small or intermediate conductances, respectively. SK_{Ca} and IK_{Ca} channels do not show any voltage dependence like the BK channel described above. SK_{Ca} channels are expressed in different neuronal tissues, in skeletal muscles, gland cells, liver cells, lymphocytes, and other peripheral cells. SK_{Ca} channels are important in mechanisms, where a specific regulation of the cellular membrane potential is required for the normal function of cells. e.g. the after-hyperpolarization in neuronal tissues influencing the firing pattern of neurons. IK_{Ca} channels are expressed, e.g. in endothel cell, red blood cells, and lymphocytes. These channels are also responsible for a tightly regulated membrane potential to guarantee a specific cellular function, e.g. the activation processes of T-lymphocytes. Other K⁺ channels that are important for a specific regulation of the membrane potential are K_{ATP} channels. These K⁺ channels belong to the subfamily of channels with 2 transmembranal segments and are inhibited by intracellular ATP. These channels are expressed, e.g. in insulin secreting cells or in vascular muscles, where they have an important role in regulating vascular tone (for review see Coghlan et al., J. Med. Chem, 44, 1627-1653 (2001).

In general, modulation of K⁺ channels by agonistic or antagonistic compounds can influence the membrane potential of K⁺-expressing cells, enabling a specific modulation of cells and/or tissues that might be useful in the treatment of diseases linked to membrane potential or conductance dependent cellular functions.

Several natural and synthetic molecules with the ability to modulate K⁺ channels have been identified in the past. Examples of such compounds are the avena pyrone with BK channel opening activity (WO 93/08800), triaminobenzene analogues were reported to show K⁺ channel opening activity (U.S. Pat. No. 5,200,422), the aryl-pyrrole NS-8 has been disclosed to act as a K⁺ channel opener useful in the treatment of bladder dysfunction (Tanaka, et al., J. Urol. 159, 21 (1998)), indole-3-carboxylic acid esters have been shown to exert BK opening activity (Hu et al, Drug.Dev.Res. 41, 10 (1997)), benzimidazole derivatives with K_{ATP} and BK opening activity (U.S. Pat. No. 5,475,015), novel compounds (eg. NS004) with K⁺ channel opening activity by Neurosearch (WO 00169838; WO 00134248) and 3-substituted oxoindole derivatives with BK-channel opening activity for neuronal protection especially after ischemic stroke (U.S. Pat. No. 5,602,169).

In general, the present invention provides compounds useful as medicaments, in particular for the treatment or alleviation of diseases, disorders, and conditions associated with potassium channels.

The present invention is therefore directed to the use of a compound of the Formula (I)

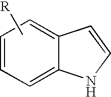

or a salt, a physiologically functional derivative, or a prodrug thereof as a medicament,

wherein

R is a monocyclic or polycyclic substituted or unsubstituted aromatic ring system which may contain one or more groups X and which contains at least one aromatic ring;

X is selected from the group consisting of S, O, N, NR′, SO or SO₂;

R is optionally substituted by one to four substituents which are independently selected from the group consisting of halogen, CF₃, OCF₃, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, arylalkyl, alkylsulfonyl, H, hydroxy, aryl, heteroaryl, —NR′OR′, CN, alkylsulfinyl, arylsulfonyl, heteroarylsulfonyl, SO₃R′, NO₂, —CO—NR′R¹, arylalkyl-O—, —O-aryl, —O-heteroaryl,- arylalkyl-S—, —S-aryl, —S-heteroaryl, —NR¹—SO₂R′, —SO₂—NR¹-alkyl, —SO₂—NR¹-aryl, and —SO₂—N¹-heteroaryl;

R′ is hydrogen, alkyl, cycloalkyl, hydroxyalkyl, haloalkyl, hydroxyalkylamine, amine, alkylamine, arylalkyl, aryl or heteroaryl;

R¹ is hydrogen, hydroxy, alkyl, cycloalkyl, hydroxyalkyl, haloalkyl, arylalkyl, aryl or heteroaryl;

R² is hydrogen, hydroxy, alkyl, cycloalkyl, hydroxyalkyl, haloalkyl, arylalkyl, aryl or heteroaryl, alkoxy;

wherein an alkyl group and the alkyl parts of the above groups denote a linear or branched chain of 1 to 6 carbon atoms which may contain one or more double bonds or one or more triple bonds and which is optionally substituted by one or more substituents R′, wherein R′ being as defined above;

an alkylsulfonyl group denotes an (SO₂)-alkyl group, the alkyl group being as defined above;

an alkylsulfinyl group denotes an (SO)-alkyl group, the alkyl group being as defined above;

a cycloalkyl group denotes a non-aromatic ring system, saturated or partially saturated, monocyclic or bicyclic carbocyclic alkyl contain 4 to 8 carbon atoms, wherein one or more of the carbon atoms in the ring can be substituted by a group X, X being as defined above, and wherein the cycloalkyl group is optionally substituted by one or more substituents R′, wherein R′ being as defined above:

an alkoxy group denotes an O-alkyl group, the alkyl group being as defined above:

an alkylthio group denotes an S-alkyl group, the alkyl group being as defined above;

a haloalkyl group denotes an alkyl group which is substituted by one to five halogen atoms, the alkyl group being as defined above;

a hydroxyalkyl group denotes an HO-alkyl group, the alkyl group being as defined above;

a haloalkyloxy group denotes an alkoxy group which is substituted by one to five halogen atoms, the alkyl group being as defined above,

a hydroxyalkylamine group denotes an (HO-alkyl)₂-N— group or HO-alkyl-NH— group, the alkyl group being as defined above,

an amine group denotes an NR¹R² group, R¹ and R² being as defined above;

an alkylamine group denotes an HN-alkyl or N-dialkyl group, the alkyl group being as defined above;

an aminoalkyl group denotes an H₂N-alkyl, monoalkylaminoalkyl, or dialkylaminoalkyl group, the alkyl group being as defined above;

a halogen group is chlorine, bromine, fluorine or iodine;

an aryl group denotes an aromatic group having 5 to 15 carbon atoms which is optionally substituted by one or more substituents R′; wherein R′ being as defined above;

an arylalkyl group denotes an alkyl group which is substituted by one to three preferably one aryl groups, the alkyl and aryl group being as defined above;

an arylsulfonyl group denotes an (SO₂)-aryl group, the aryl group being as defined above;

a heteroaryl group denotes a 5- or 6-membered heterocyclic group which contains at least one heteroatom O, N, or S, which is optionally fused to another ring and the heterocyclic group is optionally substituted by one or more substituents R′, wherein R′ being as defined above;

a heteroarylsulfonyl group denotes an (SO₂)-heteroaryl group, the heteroaryl group being as defined above;

The invention also provides a pharmaceutical composition comprising a compound of Formula (I), in free form or in the form of pharmaceutically acceptable salts or physiologically functional derivatives together with a pharmaceutically acceptable diluent or carrier therefore.

The term “physiologically functional derivative” as used herein refers to compounds which are not pharmaceutically active themselves but which are transformed into their pharmaceutically active form in vivo, i.e. in the subject to which the compound is administered. The physiologically functional derivative may be an ester, amide or sulfamide derivative of the compound of Formula (I) or of a salt thereof.

In another aspect, the present invention also provides a method for the treatment or prophylaxis of a condition where there is an advantage in regulating the membrane potential and/or conductance in cells of mammals, including a human, by the specific modulation of potassium channels which comprises the administration of an effective amount of a compound of Formula (I) and physiologically acceptable salts or physiologically functional derivatives thereof.

The invention is also directed to the use of compounds of the Formula (I) and of their pharmacologically tolerable salts or physiologically functional derivatives for the production of a medicament for the prevention, alleviation and/or treatment of diseases in mammals, including a human, responsive to the specific modulation of potassium channels.

In addition, the present invention provides methods for preparing the desired indole of the Formula (I).

A first method for synthesis of the arylindole of the Formula (I) comprises the step of reacting an arylhalide [T. Oh-e, N. Miyaura, A. Suzuki, J. Org. Chem. (1993), 58, 2201-2208; W. A. Herrmann, V. P. W. Böhm, C. -P Reisinger, J. Organomet. Chem. (1999), 576, 23-41; S. P. Stanforth, Tetrahedron (1998), 54, 263-303; N. Miyaura, A. Suzuki, Chem. Rev. (1995), 95, 2457-2483; A. Suzuki, J. Organomet. Chem. (1999), 576, 147-168; A. Bahl, W. Grahn, S. Stadler, F. Feiner, G. Bourhill, C. Br\overline{a}uchle, A. Reisner, P. G. Jones, Angew. Chem. Int. Ed. Engl. (1995), 34, 1485-1488.] or aryltriflate of Formula (II) with an aryl boronic acid of the Formula (III) [T. Oh-e, N. Miyaura, A. Suzuki, J. Org. Chem. (1993), 58, 2201-2208).

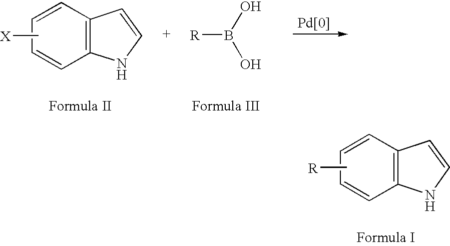

A second method of the invention for preparing the compounds of Formula (I) comprises the step of reacting an indole boronic acid of the Formula (IV) with an arylhalide or aryltriflate of the general Formula (V).

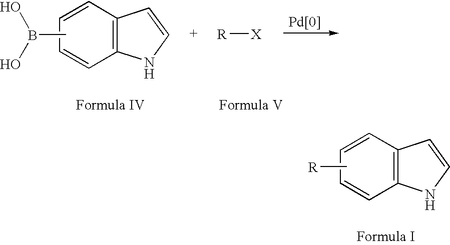

In a preferred embodiment of the invention, R is an aromatic mono- or bicyclic hydrocarbon group having 5 to 15 carbon atoms, in particular having 5 to 10 carbon atoms, which optionally contains 1 to 4 N and/or O and/or S heteroatoms, in particular by 1 to 3 of these heteroatoms. Preferably, R is selected from a phenyl, furan, thiophene, oxazole, thiazole, isoxazole, isothiazole, 1,2,3-triazole, 1,3,4-thiadiazole, pyran, indole, isoindole, pyridine, pyridazine, pyrimidine, pyrazine, indazole, benzimidazole, triazine, indolizine, benzofuran, benzothiophene, benzothiophene-1,1-dioxide, benzothiazole, purine, quinolizine, quinoline, isoquinoline, cinnoline, phthalazine, quinazoline, naphthyridine, benzodioxol, naphthalin, and pteridine group. Particularly preferred compounds are those in which R is a phenyl group or thiophene, benzodioxol, naphthalin.

One or more of the carbon atoms in the ring system R can be substituted by a group X, wherein X is selected from the group consisting of S, O, N, NR′, SO or SO₂. In one preferred embodiment, one of the carbon atoms is substituted by a group X.

In other preferred embodiments, optional substituents of R are Cl, OCH₃, or C₁-C₅-alkyl ethinyl, phenyl, NO₂, —NHCOCH₃, —COCH₃, —COCH₂CH₃, —SO₂CH₃, —COH, —C(CH₃)₃, —N(CH₃)₂, preferably F, CF₃, OCF₃, ethinyl phenyl, NO₂, —NHCOCH₃, —COCH₃, —COCH₂CH₃, —SO₂CH₃, —CHO, —C(CH₃)₃, —N(CH₃)₂.

In a preferred embodiment of the invention, R is a phenyl group in the 4-position to the indol group of the compound of the Formula (I) and one or more substituents of R are in ortho-, meta-, or para-position of the phenyl group and preferably represent halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, most preferably F, CF₃, OCF₃, Cl, ethinyl, phenyl, H, NO₂, —NHCOCH₃, —COCH₃, —COCH₂CH₃, —SO₂CH₃, —CHO, —C(CH₃)₃, —N(CH₃)₂,

In another preferred embodiment of the invention, R is a phenyl group in the 5-position to the indol group of the compound of the Formula (I) and one or more substituents of R are in ortho-, meta-, or para-position of the phenyl group and the substituents of R are preferably halogen, CF₃, OCF₃, alkyl, cycloalkyl, haloalkyl, haloalkyloxy, hydroxyalkyl, hydroxyalkylamine, amine, aminoalkyl alkylamine, CR′O, CO₂R′, alkoxy, alkylthio, most preferably F, CF₃, OCF₃, Cl, ethinyl, phenyl, NO₂, —NHCOCH₃, —COCH, —COCH₂CH₃, —SO₂CH₃, —CHO, —C(CH₃)₃, —N(CH₃)₂.

In another preferred embodiment of the invention, R is a phenyl group in the 6-position to the indol group of the compound of the Formula (I) and one or more substituents of R are in ortho-, meta-, or para-position of the phenyl group and the substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, most preferably F, CF₃, OCF₃, Cl, ethinyl, phenyl, NO₂, —NHCOCH₃, —COCH₃, —COCH₂CH₃, —SO₂CH₃, —CHO, —C(CH₃)₃, —N(CH₃)₂.

In another preferred embodiment of the invention, R is a phenyl group in the 7-position to the indol group of the compound of the Formula (I) and one or more substituents of R are in ortho-, meta-, or para-position of the phenyl group and the substituents of R are preferably halogen, alkyl, cycloalkyl, haloalkyl, haloalkyloxy, hydroxyalkyl hydroxyalkylamine, amine, aminoalkyl, alkylamine, CR′O, CO₂R′, alkoxy, alkylthio, most preferably F, CF₃, OCF₃, Cl, ethinyl, phenyl, NO₂, —NHCOCH₃, —COCH₃, —COCH₂CH₃, —SO₂CH₃, —CHO, —C(CH₃)₃, —N(CH₃)₂.

In another preferred embodiment of the invention, R is a thienyl moiety in the 4-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂, most preferably F, CF₃, OCF₃, Cl.

In another preferred embodiment of the invention, R is a thienyl moiety in the 5-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is a thienyl moiety in the 6-position to the indol group of the compound of the Formula (I) and one or more substituents of R are preferably halogen, alkyl, cycloalkyl, haloalkyl, haloalkyloxy, hydroxyalkyl hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is a thienyl moiety in the 7-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is a naphthyl moiety in the 4-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃. —N(CH₃)₂, most preferably F, CF₃, OCF₃, Cl.

In another preferred embodiment of the invention, R is a naphthyl moiety in the 5-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is a naphthyl moiety in the 6-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is a naphthyl moiety in the 7-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is an indolyl moiety in the 4-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is an indolyl moiety in the 5-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is an indolyl moiety in the 6-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is an indolyl moiety in the 7-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, Cl.

In another preferred embodiment of the invention, R is a benzodioxolyl moiety in the 4-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl NO₂, —NHCOCH₃, —COCH₃, —COCH₂CH₃, —SO₂CH₃, —CHO, —C(CH₃)₃, —N(CH₃)₂ most preferably F, CF₃, OCF₃.

In another preferred embodiment of the invention, R is a benzodioxolyl moiety in the 5-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′, CO₂R′, alkoxy, alkylthio, Cl, ethinyl, phenyl NO₂, —NHCOCH₃, —COCH₃, —COCH₂CH₃, —SO₂CH₃, —CHO, —C(CH₃)₃, —N(CH₃)₂ most preferably F, CF₃, OCF₃.

In another preferred embodiment of the invention, R is a benzodioxolyl moiety in the 6-position to the indol group of the compound of the Formula (I) and one or more substituents of R are preferably halogen, alkyl, cycloalkyl, haloalkyl haloalkyloxy, hydroxyalkyl, hydroxyalkylamine, amine, aminoalkyl, alkylamine, CR′O, CO₂R′, alkoxy, alkylthio, Cl, ethinyl, phenyl NO₂, —NHCOCH₃, —COCH₃, —COCH₂CH₃, —SO₂CH₃, —CHO, —C(CH₃)₃, —N(CH₃)₂ most preferably F, CF₃, OCF₃.

In another preferred embodiment of the invention, R is a benzodioxolyl moiety in the 7-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl NO₂, —NHCOCH₃, —COCH₃, —COCH₂CH₃, —SO₂CH₃, —CHO, —C(CH₃)₃, —N(CH₃)₂ most preferably F, CF₃, OCF₃.

In another preferred embodiment of the invention, R is a pyridinyl moiety in the 4-position to the indol group of the compound of the Formula (I) and one or more substituents of R are preferably halogen, alkyl, cycloalkyl, haloalkyl, haloalkyloxy, hydroxyalkyl hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, —OCH₃, Cl.

In another preferred embodiment of the invention, R is a pyridinyl moiety in the 5-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl. ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, —OCH₃, Cl.

In another preferred embodiment of the invention, R is a pyridinyl moiety in the 6-position to the indol group of the compound of the Formula (I) and one or more substituents of R are preferably halogen, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, —OCH₃, Cl.

In another preferred embodiment of the invention, R is a pyridinyl moiety in the 7-position to the indol group of the compound of the Formula (I) and one or more substituents of R are preferably halogen, alkyl, cycloalkyl, haloalkyl, haloalkyloxy, hydroxyalkyl hydroxyalkylamine, amine, aminoalkyl, alkylamine, CR′O, CO₂R′, alkoxy, alkylthio, Cl, ethinyl, phenyl, NO₂, —NHCOCH₃, —COCH₃, —COCH₂CH₃, —SO₂CH₃, —CHO, —C(CH₃)₃, —N(CH₃)₂ most preferably F, CF₃, OCF₃, —OCH₃, Cl.

In another preferred embodiment of the invention, R is a phenyl moiety in the 6-position to the indol group of the compound of the Formula (I) and one or more substituents of R are preferably unsubstituted or substituted phenyl.

Most preferred compounds and/or pharmaceutically acceptable salts thereof, selected from the group comprising:

5-(2-Nitrophenyl)-1H-indole; 5-Phenyl-1H-indole; 5-(2-Methoxyphenyl)-1H-indole; 6-Benzofuran-2-yl-1H-indole; 6-(3-Trifluormethoxyphenyl)-1H-indole, 6-(6Methoxy-pyridin-3-yl)-1H-indole, [4(1H-Indol-6-yl)-phenyl]-dimethylamine: 7-Naphthalen-2-yl-1H-indole; 4-(4-Trifluoromethylphenyl)-1H-indole; 7-(6-Methoxypyridin-3-yl)-1H-indole; 7-(4-tert-Butylphenyl)-1H-indole; 4-(4-Benzyloxyphenyl)-1H-indole; 6-(4-Trifluoromethylphenyl)-1H-indole; [4(1H-Indol-7-yl)-phenyl]-dimethyl-amine: 6-(3-Fluoro-phenyl)-1H-indole; 7-(3-Trifluormethoxyphenyl)-1H-indole; 4-(4-tert-Butylphenyl)-1H-indole; [4(1H-Indol-4yl)-phenyl]-dimethyl-amine; N-[3-(1H-Indol-7-yl-phenyl]-acet-amide; 6-Naphthalen-2-yl-1H-indole; 4-(4-tert-Butylphenyl)-1H-indole; 4-(4-Methoxy-phenyl)-1H-indole; 6-(4-Trifluoromethoxyphenyl)-1H-indole; 4-(4-Trifluoromethoxy-phenyl)-1H-indole; 1-[2-(1H-Indol-5-yl)-phenyl]-ethanone; 1H,1′H-[5,5′]Biindolyl; 5-Naphthalen-1-yl-1H-indole; 5-(4-Trifluoromethylphenyl)-1H-indole; 5-(4-Ethynyl-phenyl)-1H-indole; 1-[4 (1H-Indol-5-yl)-phenyl]-propan-1-one; 5-(4-Chloro-phenyl)-1H-indole; 1H,1′H-[4,5′]Biindolyl; 5-(2,4-Dimethoxy-phenyl)-1H-indole; 5-(4-Trifluoromethoxyphenyl)-1H-indole; 1-[3-(1H-Indol-5-yl)-phenyl]-ethanone: 5-Benzo[1,3]dioxol-5-yl-1H-indole; 5-(4-Methoxy-3,5-dimethylphenyl)-1H-indole; 5-Pyridin-2-yl-1H-indole; 1H,1′-[5,6′]Biindolyl; 5-(4-Methoxyphenyl)-1H-indole; 6-(4-Phenoxyphenyl)-1H-indole; 6-Biphenyl-3-yl-1H-indole; 7-(1H-Indol-5-yl)-4methyl-indan-1-one; 7-Benzo-furan-2-yl-1H-indole; 4-(2,4-Difluorophenyl)-1H-indole; 4-Biphenyl-3-yl-1H-indole; 4(3-Methoxyphenyl)-1H-indole; 4-(4-Phenoxyphenyl)-1H-indole; 6-(3,5-Bistrifluoromethyl-phenyl)-1H-indole; 6-(4-Chlorophenyl)-1H-indole; 7-(4-Phenoxyphenyl)-1H-indole; 7-Bi-phenyl-3-yl-1H-indole; 6-(2,4-Difluorophenyl)-1H-indole; [4(1H-Indol-5-yl-phenyl]-dimethylamine; 5-(2,4Difluorophenyl)-1H-indole; 7-(5-Chlorothiophen-2-yl)-1H-indole; 7-(2-Methoxyphenyl-1H-indole; 7-(2-Chlorophenyl)-1H-indole; 6-(5-Chlorothiophen-2-yl)-1H-indole; 6-(2-Chlorophenyl)-1H-indole; 6-(2-Methoxyphenyl)-1H-indole; 4-(3,5-Bis-trifluoromethylphenyl)-1H-indole; 4-(2-Fluorobiphenyl-4yl)-1H-indole, 4(5-Chloro-thiophen-2-yl)-1H-indole; 4-Thiophen-3-yl-1H-indole; 4(2-Methoxyphenyl)-1H-indole; 5-(4-Methanesulfonylphenyl)-1H-indole; 5-(2-Chloro-phenyl)-1H-indole; 2-(1H-Indol-5-yl)-benzaldehyde; 5-(4tert-Butylphenyl)-1H-indole.

The compounds of the Formula (I) to be used according to the invention can form salts with inorganic or organic acids or bases. Examples of such salts are, for example ammonium salts.

An alkyl group, if not stated otherwise, is preferably a linear or branched chain of 1 to 6 carbon atoms, preferably a methyl ethyl n-propyl, iso-propyl, n-butyl, tert-butyl, iso-butyl, n-pentyl, 2-dimethylbutyl or n-hexyl group, a methyl, ethyl, iso-propyl or tert-butyl group being most preferred.

An alkyl group includes moreover a linear or branched chain of 1 to 6 carbon atoms, having one or more double bonds or one or more triple bonds, preferably 1 to 2 double or 1 to 2 triple bonds and more preferably one double/triple bond, preferably an allyl, ethenyl, propenyl, 2-methylpropenyl, 1,4butadienyl, ethinyl, propinyl, iso-prenyl, hexa-2-enyl, and the like.

The C₁-C₆-alkyl residue may be selected from the group comprising —CH₃, —C₂H₅, —CH═CH₂, C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, —C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—CH≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—CH—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄h₉, —C₃H₆—CH═CH—CH₃, —CH₂—CH═CH—C₃h₇, —C₂H₄—CH═CH—C₂H₅, —CH₂C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅;

The alkyl group in the compounds of formula (I) is optionally substituted by one or more substituents R′, wherein R′ being as defined above, preferably by halogen.

An alkylsulfonyl group denotes an (SO₂)-alkyl group, the alkyl group being defined above. An alkylsulfonyl group can include, but is not limited to, methylsulfonyl, ethylsulfonyl, n-propylsulfonyl, iso-propylsulfonyl, n-butylsulfonyl, iso-butylsulfonyl, sec-butylsulfonyl, tert-butylsulfonyl, and the like.

An alkylsulfinyl group denotes an (SO)-alkyl group, the alkyl group being as defined above. An alkylsulfinyl group can include, but is not limited to, methylsulfinyl, ethylsulfinyl, n-propylsulfinyl, iso-propylsulfinyl, n-butylsulfinyl, iso-butylsulfinyl, sec-butylsulfinyl, tert-butylsulfinyl, and the like.

An cycloalkyl group denotes a non-aromatic ring system, saturated or partially saturated, monocyclic or bicyclic carbocyclic alkyl, containing 4 to 8 carbon atoms, wherein the ring system comprises one or more of the carbon atoms in the ring can be substituted by a group X, X being as defined above. The cycloalkyl group is optionally substituted by one or more substituents R′, wherein R′ being as defined above. A cycloalkyl group can include, but is not limited to, cyclopentyl, cyclohexyl, cyclohex-2enyl, dihydroxycyclohexyl, cycloheptyl, tetraline, and the like.

An alkoxy group denotes an O-alkyl group, the alkyl group being as defined above. An alkoxy group can include, but is not limited to, methoxy, ethoxy, n-propoxy, iso-propoxy, n-butoxy, iso-butoxy, sec-butoxy, tert-butoxy, and the like.

An alkylthio group denotes an S-alkyl group, the alkyl group being as defined above. An alkylthio group can include, but not is limited to, methylthio, ethylthio, n-propylthio, iso-propylthio, n-butylthio, iso-butylthio, sec-butylthio, tert-butylthio, and the like.

An haloalkyl group denotes an alkyl group which is substituted by one to five preferably three halogen atoms, the alkyl group being as defined above. A haloalkyl group can include, but is not limited to, 1,1,1-trifluoroethyl, chloromethyl, 1-bromoethyl, fluoromethyl, difluoromethyl, trifluoromethyl, and the like.

A hydroxyalkyl group denotes an HO-alkyl group, the alkyl group being as defined above. A hydroxyalkyl group can include, but not is limited to, hydroxymethyl, 2-hydroxyethyl, 3-hydroxypropyl, 2-ethyl4-hydroxyhexyl and the like.

An haloalkyloxy group denotes an alkoxy group which is substituted by one to five preferably three halogen atoms, the alkyl group being as defined above. An haloalkyloxy group can include, but is not limited to, trifluoromethoxy, 2-chloroethoxy, difluoromethoxy, 1,2-difluoroethoxy, 2,2,2-trifluoroethoxy, and the like.

A hydroxyalkylamine group denotes an (HO-alkyl)₂-N— group or HO-alkyl-NH— group, the alkyl group being as defined above. A hydroxyalkylamino group can include, but is not limited to, (HOCH₂)₂—N—, (HOC₃H₆)₂—N—, (HOC₄H₈)₂—N—, HO—CH₂—NH—, HO—C₂H₄—NH—, HO—C₃H₆—NH—, HO—C₄H₈—NH—.

An amine group denotes an NR¹R² group, R¹ and R² being as defined above.

An alkylamine group denotes an —NH— alkyl or —N-dialkyl group, the alkyl group being as defined above. An alkylamino group can include, but is not limited to, —NH—CH₃, —NH—C₂H₅, —NH—C₃H₇, —NH—C₄H₉, —NH—CH(C₂H₆), —NH—C(C₃H₉), —N—(CH₃)₂, —N—(C₂H₆)₂, —N—(C₃H₇)₂, —N—(C(C₃H₉))₂, and the like.

An aminoalkyl group denotes an H₂N-alkyl, monoalkylaminoalkyl, or dialkylaminoalkyl group, the alkyl group being as defined above. An aminoalkyl group can include, but is not limited to, H₂N—CH₂—, H₂N—CH₂-CH₂—, CH₃—NH—CH₂—, (CH₃)₂—N—CH₂, (CH₃)₂—NH—CH₂—, and the like.

A halogen group is chlorine, bromine, fluorine or iodine, fluorine being preferred.

An aryl group preferably denotes an aromatic group having 5 to 15 carbon atoms, in particular a phenyl group. This aryl group can optionally be substituted by one or more substituents R′, where R′ being as defined above, preferably by haloalkyloxy, hydrogen, alkyl, cycloalkyl, hydroxyalkyl, hydroxyalkylamine, amine, alkylamine, arylalkyl, aryl or heteroaryl. An aryl group can include, but is not limited to, phenyl, tolyl, 2-methoxyphenyl, 2-fluorophenyl, trifluorophenyl, 2-chlorophenyl, 2-nitrophenyl, aminophenyl, 3-acetamidophenyl, 3-trifluoromethoxyphenyl, 4-phenyldimethylamine, 2,4-dimethoxyphenyl naphthyl, [1.3]benzodioxol, biphenyl, phenanthryl, and the like.

An arylalkyl group denotes an alky group which is substituted by one to three preferably one aryl groups, the alkyl and aryl group being as defined above. An arylalkyl group can include, but is not limited to, benzyl, 1-phenylethyl, 2-phenylethyl dibenzylmethyl, methylphenylmethyl, diphenylmethyl, dichlorophenylmethyl, 4methoxyphenylmethyl and the like.

An arylsulfonyl group denotes an (SO₂)-aryl group, the aryl group being as defined above. An arylsulfonyl group can include, but is not limited to, C₆H₅—SO₂—, and the like.

A heteroaryl group denotes a 5- or 6-membered heterocyclic group which contains at least one heteroatom like O, N, S. This heterocyclic group can be fused to another ring. For example, this group can be selected from an oxazol-2-yl, oxazol-4yl, oxazol-5-yl, thiazol-2-yl, thiazol-4-yl, thiazol-5-yl, isothiazol-3yl, isothiazol-4yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4yl, 1,2,5-thiadiazol-3-yl, 1-imidazolyl, 2-imidazolyl, 1,2,5-thiadiazol-4-yl, 4-imidazolyl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyrimidinyl, 4-pyrimidinyl, 5-pyrimidinyl, 3-pyridazinyl, 4-pyridazinyl, 2-pyrazinyl, 1-pyrazolyl, 3-pyrazolyl, 4pyrazolyl, indolyl, indolinyl, benzo-[b]-furanyl, benzo[b]thiophenyl, benzimidazolyl, benzothiazolyl, quinazolinyl, quinoxazolinyl, or preferably isoxazol-3-yl, isoxazol-4-yl, isoxazol-5-yl, quinolinyl, tetrahydroquinolinyl, isoquinolinyl, tetrahydroisoquinolinyl [1.3]benzodioxol, anthryl, phenanthryl, fluorenyl, azulenyl, naphthyl, indenyl, phenazinyl, acridinyl, carbazolyl, pteridinyl, 1,8-naphthyridinyl, phthalazinyl, indazolyl, purinyl, quinolizinyl, indolizinyl, isoindolyl, 1,2,3-triazolyl, benzisoxazolyl, benzooxadiazolyl, benzfurazanyl, benzopyranyl, benzothiopyranyl group. This heterocyclic group can optionally be substituted by one or more substituents R′, where R′ being as defined above.

A heteroarylsulfonyl group denotes an (SO₂)-heteroaryl group, the heteroaryl group is being as defined above. A heteroarylsulfonyl group can include, but is not limited to, (SO₂)-furanyl, (SO₂)-thienyl, (SO₂)-pyridinyl, and the like.

A substituted or unsubstituted monocyclic or polycyclic aromatic ring system contains at least one aromatic ring system which can be fused to another substituted or unsubstituted ring system selected from the group of aryl or heteroaryl. The term monocyclic or polycyclic aromatic ring system mean a 5 to 15 membered monocyclic, bicyclic or tricyclic ring. This group can include, but is not limited to, naphthyl, [1.3]benzodioxol, phenanthryl, and the like. The aryl and heteroaryl groups are being as defined above.

In general, the compounds of the present invention will be useful in the treatment of disorders of a living animal body, including a human, due to their potent potassium channel modulating properties.

Therefore, the compounds of the instant invention will be useful in treating disorders of mammals, including humans, where the modulation of the membrane potential or ion conductances is influencing the effects of the disorders. Such disorders include asthma cystic fibrosis, obstructive pulmonary disease, convulsions, vascular spasms, urinary incontinence, urinary instability, urinary urgency, bladder spasms, ischemia, cerebral ischemia, traumatic brain injury, neurodegeneration, migraine, pain, psychosis, hypertension, epilepsy, memory and attention deficits, functional bowel disorders, erectile dysfunction, immune suppression, autoimmune disorders, dysfunction of cellular proliferation, diabetes, premature labour, depression, shizophrenia, sleep disorders, other forms of headache, antipsychotic and other disorders associated with or responsive to the modulation of potassium channels.

The invention provides a pharmaceutical formulation comprising a compound of Formula (I) of the invention or a pharmaceutically acceptable salt or derivative thereof, together with one or more pharmaceutically acceptable carriers therefore, and optionally, other therapeutic and/or prophylactic ingredients. The carrier(s) must be ‘acceptable’ in the sense of being compatible with the other ingredients of the formulation and not deleterious to the recipient thereof.

Pharmaceutical formulations include those suitable for oral rectal, nasal, topical (including buccal and sub-lingual), vaginal or parenteral (including intramuscular, sub-cutaneous, intradermal, and intraveneous) administration or in a form suitable for administration by inhalation or insufflation. The compounds of the invention, together with a conventional adjuvant, carrier, or diluent, may thus be placed into the form of pharmaceutical compositions and unit dosages thereof, and in such form may be employed as solids, liquids or in the form of sterile injectable solutions. If a solid carrier is used, the preparation may be tableted, placed in a hard gelatine capsule in powder or pellet form, or in form of a troche or lozenge. The solid carrier may contain conventional excipients such as binding agents, tableting lubricants, fillers, disintegrants, wetting agents and the like. Tablets may be film coated by conventional techniques. If a liquid carrier is employed, the preparation may be in form of a syrup, emulsion, soft gelatine capsule, sterile vehicle for injection, an aqueous or non-aqueous liquid suspension, or may be a dry product for reconstitution with water or other suitable vehicles before use. Liquid preparations may contain conventional additives such as suspending agents, emulsifying agents, wetting agents, non-aqueous vehicle (including edible oils), preservatives, as well as flavouring and/or colouring agents. For parenteral administration, a vehicle normally will comprise sterile water, at least in large part although saline solutions, glucose solutions and like may be utilized. Injectable suspensions also may be used, in which case conventional suspending agents may be employed. Conventional preservatives, buffering agents and the like also may be added to the parenteral dosage forms. Administration, however, can also be carried out rectally, e.g., in the form of suppositories, or vaginally, e.g. in the form of pessaries, tampons, creams, or percutaneously, e.g., in the form of ointments, creams or tinctures. Administration directly to the nasal cavity by conventional means can be carried out e.g. by pipette, spray or dropper, administration to the respiratory tract may be achieved by means of an aerosol formulation, e.g. where the active ingredient is provided in a pressurized pack with a suitable propellant, or other suitable application mechanisms.

The pharmaceutical compositions are prepared by conventional techniques appropriate to the desired preparation containing appropriate amounts of the active ingredient, that are, the compounds in this invention. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

A suitable dose of compounds or pharmaceutical compositions thereof for a mammal, especially humans, suffering from, or likely to suffer from any condition as described herein is an amount of active ingredient from about 0.1 μg/kg to 500 mg/kg body weight. For parenteral administration, the dose may be in the range of 0.1 μg/kg to 100 mg/kg body weight for intravenous administration. The active ingredient will preferably be administered in equal doses from one to four times daily. The compounds of Formula (I) can also be used in the form of a precursor (prodrug) or a suitably modified form that releases the active compound in vivo. Normally, the administered dose will be gradually increased until the optimal effective dosage for the treated host is determined The optimal administered dosage will be determined by a physician or others skilled in the art, depending on the relevant circumstances including the condition to be treated, the choice of compound to be administered, the route of administration, the sex, age, weight, and the specific response of the treated individual in respect to the severity of the individual's symptoms.

### EXAMPLES

1. Synthesis of Compounds of Formula (I)

First Method of Synthesis:

4-, 5-, 6- or 7-haloindole, or 4-,5-,6- or 7-triflateindole (100 mg, 1 eq), aryl- or heteroaryl-boronic acid (1.2 eq), Pd(PPh₃)₄ (0.03 eq), and barium hydroxide octahydrate (3.3 eq) were dissolved in a mixture of toluene (2 ml)—ethanol (2 ml)—water (1 ml). The mixture was stirred for 16 h at 100° C. and then cooled to room temperature [TLC (n-hexane-EtOAc, 8:2)]. The crude product was purified either directly by preparative thin layer chromatography (Merck, 20×20 cm, Silica gel 60 F₂₅₄, 1 mm) using (n-hexane:EtOAc, 9:1) as eluent, or filtered through a pad of celite, concentrated, and then purified by preparative thin layer chromatography [A. Suzuki, J. Organomet. Chem. (1999), 576, 147-168; A. Bahl, W. Grahn, S. Stadler, F. Feiner, G. Bourhill, C. Bräuchle, A. Reisner, P. G. Jones, Angew. Chem. Int. Ed Engl. (1995), 34, 1485-1488].

Second Method of Synthesis:

4-, 5-, 6- or 7-indole boronic acid (1,2 eq), haloaryl or heterohaloaryl or aryltriflate (100 mg, 1 eq), Pd(PPh₃)₄ (0.03 eq), and barium hydroxide octahydrate (3.3 eq) were dissolved in a mixture of toluene (2 ml)—ethanol (2 ml)—water (1 ml). The mixture was stirred for 16 h at 100° C. and then cooled to room temperature [TLC (n-hexane-EtOAc, 8:2)]. The crude product was purified either directly by preparative thin layer chromatography (Merck, 20×20 cm, Silica gel 60 F₂₅₄, 1 mm) using (n-hexane:EtOAc, 9:1) as eluent, or filtered through a pad of celite, concentrated, and then purified by preparative thin layer chromatography.

[A. Suzuki J. Organomet. Chem. (1999), 576, 147-168; A. Bahl. W. Grahn, S. Stadler, F. Feiner, G. Bourhill, C. Bräuchle, A Reisner, P. G. Jones, Angew. Chem. Int. Ed. Engl. (1995),34, 1485-1488].

Table I: Mass was determined by mass spectrometry, the exact molecular mass, the NMR data (abbreviations: br.=broad, s=singulet, d=doublet, t=triplet, m=multiplet, J=¹H-³H coupling constant) and the Eₘ assay results are shown. Eₘ Assay results are given as the ratio of the compound effect (50 μM) compared to the maximal effect of NS004 (25 or 50 μM. Ranges are 0-1=+, >1=++, blocking effects (showing increased fluorescence intensity) are given as —.

| | | HPLC/MS | | Eₘ |
|---|---|---|---|---|
| N | Structure | (ESI) | ¹H-NMR (300 MHz) | effect |
| 1 | 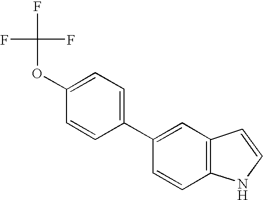 | 278 [M + H]^{+276 [M - H]}⁺ | δ (CDCl₃) = 6.52 (s, 1 H, H-3), 7.14-7.20 (m, 3 H, H-2, H-3′and H-5′), 7.29-7.37 (m, 2 H, H-6 and H-7), 7.54 (d, J = 8.4 Hz, 2 H, H-2′ and H-6′), 7.73 (s, 1 H, H-4), 8.04 (s, 1 H, NH) | ++ |
| 2 | 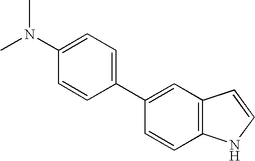 | 237 [M + H]⁺ | δ (CDCl₃) = 2.92 (s, 6 H, N(CH₃)₂), 6.50 (br. s, 1 H, H-3), 6.85 (d, J = 7.3 Hz, 2 H, H-3′and H-5′), 7.14 (br. s, 1 H, H-2), 7.34 (s, 2 H, H-6 and H-7), 7.49 (d, J = 8.4 Hz, 2 H, H-2′and H-6′), 7.72 (s, 1 H, H-4), 8.08 (s, 1 H, NH) | + |
| 3 | 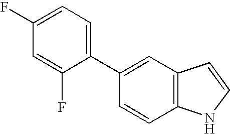 | 230 [M + H]^{+228 [M + H]}⁺ | δ (CDCl₃) = 6.51 (br. s, 1 H, H-3), 6.79-6,89 (m, 2 H, H-3′ and H-5′), 7.16 (br. s, 1 H, H-2), 7.25 (d, J = 8.4 Hz, 1 H, H-6′), 7.35-7.39 (m, 2 H, H-6 and H-7), 7.68 (s, 1 H, H-4), 8.10 (s, 1 H, NH) | ++ |
| 4 | 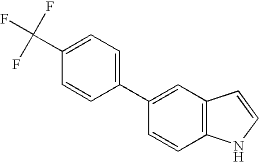 | 262 [M + H]^{+260 [M - H]}⁺ | δ (CDCl₃) = 6.54 (br. s, 1 H, H-3), 7.18 (br. s, 1 H, H-2), 7.38 (s, 2 H, H-6 and H-7), 7.59 (d, J = 8.7 Hz, 2 H, H-2′ and H-6′), 7.66 (s, J = 8.3 Hz, 2 H, H-3′and H-5′), 7.79 (s, 1 H, H-4), 8.12 (s, 1 H, NH) | ++ |
| 5 | 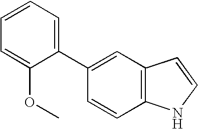 | 224 [M + H]⁺ | δ (CDCl₃) = 3.71 (s, 3 H, OCH₃), 6.89-6,97 (m, 2 H, H-3′and H-5′), 7.07 (br. s, 1 H, H-2), 7.20 (t, J = 6.9 Hz, 1 H, H-4′), 7.29-7.31 (m, 3 H, H-6, H-7 and H-6′), 7.69 (s, 1 H, H-4), 7.99 (s, 1 H, NH) | ++ |
| 6 | 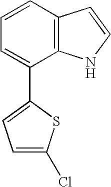 | 234 [M + H]^{+232 [M - H]}⁺ | | + |
| 7 | 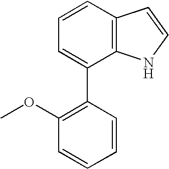 | 224 [M + H]⁺ | | + |
| 8 | 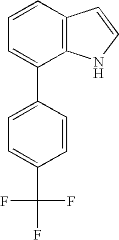 | 262 [M + H]^{+260 [M - H]}⁺ | | + |
| 9 | 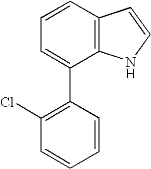 | 228 [M + H]⁺ | | + |
| 10 | 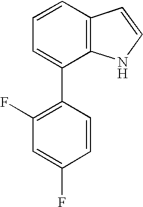 | 230 [M + H]^{+228 [M - H]}⁺ | | + |
| 11 | 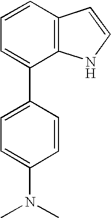 | 237 [M + H]⁺ | | ++ |
| 12 | 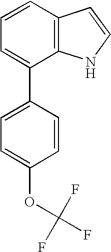 | 278 [M + H]^{+276 [M - H]}⁺ | | − |
| 13 | 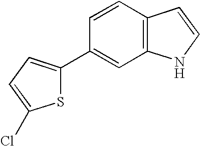 | 234 [M + H]^{+232 [M - H]}⁺ | | + |
| 14 | 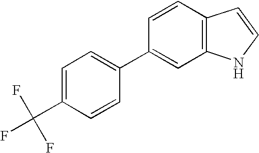 | 262 [M + H]^{+260 [M - H]}⁺ | | + |
| 15 | 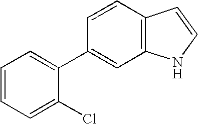 | 228 [M + H]⁺ | | + |
| 16 | 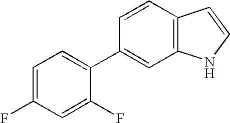 | 230 [M + H]^{+228 [M - H]}⁺ | | + |
| 17 | 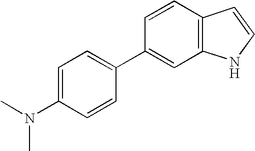 | 237 [M + H]⁺ | | ++ |
| 18 | 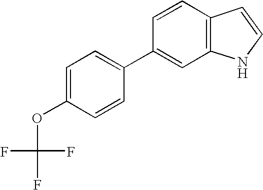 | 278 [M + H]^{+276 [M - H]}⁺ | | − |
| 19 | 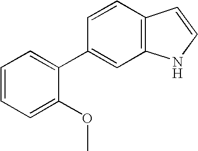 | 224 [M + H]⁺ | | + |
| 20 | 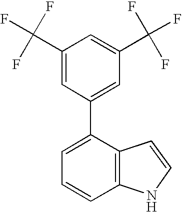 | 330 [M + H]^{+328 [M - H]}⁺ | | + |
| 21 | 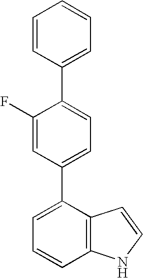 | 288 [M + H]^{+286 [M - H]}⁺ | | + |
| 22 | 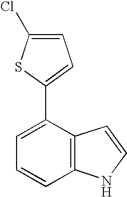 | 234 [M + H]^{+232 [M - H]}⁺ | | + |
| 23 | 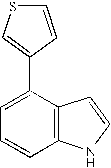 | 200 [M + H]^{+198 [M - H]}⁺ | | + |
| 24 | 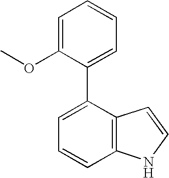 | 224 [M + H]⁺ | | + |
| 25 | 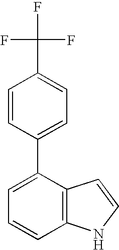 | 262 [M + H]^{+260 [M - H]}⁺ | | + |
| 26 | 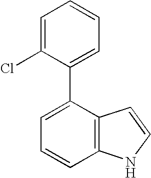 | 228 [M + H]^{+226 [M - H]}⁺ | | + |
| 27 | 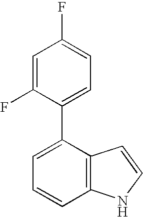 | 230 [M + H]^{+228 [M - H]}⁺ | | + |
| 28 | 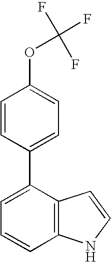 | 278 [M + H]^{+276 [M - H]}⁺ | | + |
| 29 | 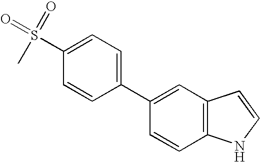 | 272 [M + H]^{+270 [M - H]}⁺ | | + |
| 30 | 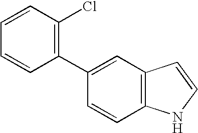 | 228 [M + H]⁺ | | ++ |
| 31 | 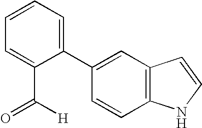 | 222 [M + H]^{+220 [M - H]}⁺ | | + |
| 32 | 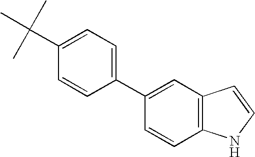 | 250 [M + H]^{+248 [M - H]}⁺ | | + |
| 31 | 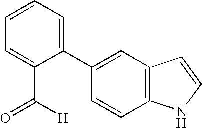 | 222 [M + H]^{+220 [M - H]}⁺ | | + |
| 32 | 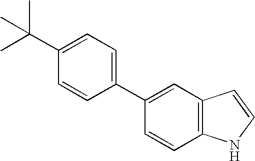 | 250 [M + H]^{+248 [M - H]}⁺ | | + |
| 33 | 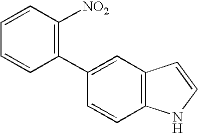 | 239 [M + H]^{+237 [M - H]}⁺ | δ (CDCl₃) = 6.46 (s, 1H, 6.99-7.03 (dd, 1H), 7.10 (t, 1H), 7.23-7.52 (m, 5H), 7.69 (dd, 1H), 8.15 (br. s, 1H) | + |
| 34 | 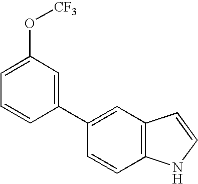 | 278 [M + H]^{+276 [M - H]}⁺ | | + |
| 35 | 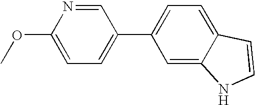 | 225 [M + H]^{+223 [M - H]}⁺ | δ (CDCl₃) = 3.99 (s, 3H), 6.58 (dt, 1H), 7.24 (dd, 1H), 7.25 (dd, 1H), 7.29 (dd, 1H), 7.53 (d, 1H), 7.69 (d, 1H), 7.83 (dd, 1H), 8.25 (br. s, 1H), 8.43 (dd, 1H) | + |
| 36 | 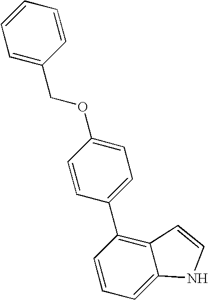 | 300 [M + H]^{+298 [M - H]}⁺ | | + |
| 37 | 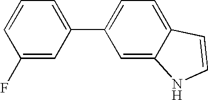 | 212 [M + H]^{+210 [M - H]}⁺ | | + |
| 38 | 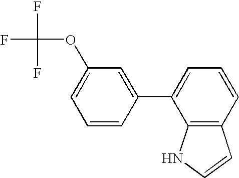 | 278 [M + H]^{+276 [M - H]}⁺ | | + |
| 39 | 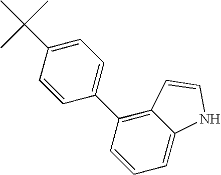 | 250 [M + H]^{+248 [M - H]}⁺ | δ (CDCl₃) = 1.39 (s, 9H), 6.76 (septuplet, 1H), 7.19 (dd, 1H), 7.20-7.29 (m, 2H), 7.37 (dt, 1H), 7.57 (quartet, 4H), 8.21 (br. s, 1H) | ++ |
| 40 | 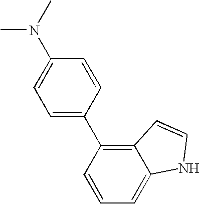 | 228 [M + H]^{+226 [M - H]}⁺ | δ (CDCl₃) = 3.02 (s, 6H), 6.76 (dt, 1H), 6.81 (s, 2H), 7.15 (dd, 1H), 7.21-7.26 (m, 2H), 7.32 (d, 1H), 7.63 (dd, 2H), 8.18 (br. s, 1H) | + |
| 41 | 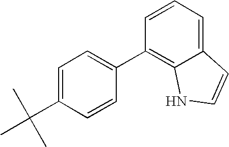 | 250 [M + H]^{+248 [M - H]}⁺ | δ (CDCl₃) = 1.39 (s, 9H), 6.61 (dt, 1H), 7.18-7.25 (m, 3H), 7.48-7.62 (m, 5H), 8.41 (br. s, 1H) | − |
| 42 | 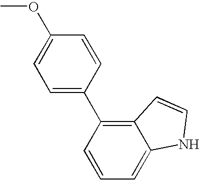 | 224 [M + H]^{+222 [M - H]}⁺ | δ (CDCl₃) = 3.87 (s, 3H), 6.71 (dt, 1H), 7.01 (d, 2H), 7.15 (dd, 1H), 7.23-7.28 (m, 2H), 7.35 (d, 1H), 7.62 (d, 2H), 8.21 (br. s, 1H) | + |
| 43 | 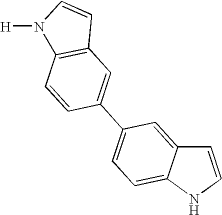 | 233 [M + H]^{+231 [M - H]}⁺ | δ (CDCl₃) = 6.60 (dt, 2H), 7.23 (t, 2H), 7.43-7.53 (m, 4H), 7.89 (dt, 2H), 8.12 (br. s, 2H) | + |
| 44 | 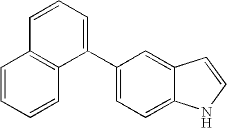 | 244 [M + H]^{+242 [M - H]}⁺ | δ (CDCl₃) = 6.54-6.62 (m, 1H), 7.21-7.54 (m, 7H), 7.75 (t, 1H), 7.85 (ddt, 1H), 7.89 (d, 1H), 7.97 (d, 1H), 8.17 (br. s, 1H) | ++ |
| 45 | 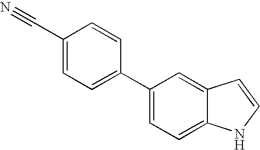 | 218 [M + H]^{+216 [M - H]}⁺ | δ (CDCl₃) = 3.10 (s, 1H), 6.61 (t, 1H), 7.20-7.42 (m, 3H), 7.58 (dd, 4H), 7.86 (s, 1H), 8.18 (br. s, 1H) | ++ |
| 46 | 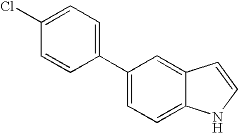 | 228 [M + H]^{+226 [M - H]}⁺ | δ (CDCl₃) = 6.58 (t, 1H), 7.17 (dd, 1H), 7.35-7.56 (m, 6H), 7.80 (d, 1H), 8.08 (br. s, 1H) | ++ |
| 47 | 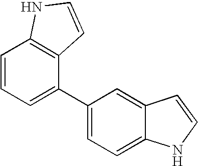 | 233 [M + H]^{+231 [M - H]}⁺ | | + |
| 48 | 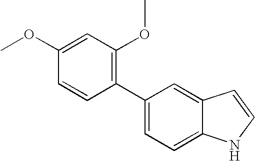 | 254 [M + H]^{+252 [M - H]}⁺ | δ (CDCl₃) = 3.78 (s, 3H), 3.85 (s, 3H), 6.55-6.58 (m, 3H), 7.19 (t, 1H), 7.24-7.40 (m, 3H), 7.72 (t, 1H), 8.10 (br. s, 1H) | ++ |
| 49 | 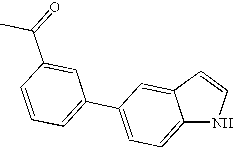 | 236 [M + H]^{+234 [M - H]}⁺ | δ (CDCl₃) = 2.64 (s, 3H), 6.59 (dt, 1H), 7.21 (t, 1H), 7.43 (d, 2H), 7.47-7.52 (m, 1H), 7.81-7.89 (m, 3H), 8.23 (t, 1H), 8.42 (br. s, 1H) | + |
| 50 | 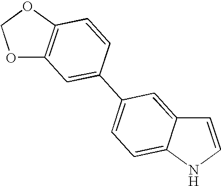 | 238 [M + H]^{+236 [M - H]}⁺ | δ (CDCl₃) = 5.98 (s, 2H), 6.57-6.59 (m, 1H), 6.88 (d, 1H), 7.08-7.13 (m, 2H), 7.22 (t, 1H), 7.34-7.43 (m, 2H), 7.76 (t, 1H), 8.13 (br. s, 1H) | ++ |
| 51 | 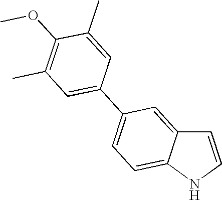 | 252 [M + H]^{+250 [M - H]}⁺ | δ (CDCl₃) = 2.36 (s, 6H), 3.77 (s, 3H), 6.58 (t, 1H), 7.20-7.22 (m, 1H), 7.29 (s, 2H), 7.40 (d, 2H), 7.80 (d, 1H), 8.12 (br. s, 1H) | ++ |
| 52 | 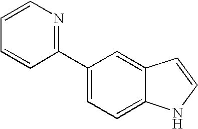 | 195 [M + H]^{+193 [M - H]}⁺ | δ (CDCl₃) = 6.60 (t, 1H), 7.14-7.19 (m, 2H), 7.41 (dt, 1H), 7.71-7.78 (m, 2H), 7.88 (d, 1H), 8.27 (d, 1H), 8.67 (br. s, 1H), 8.70 (d, 1H) | + |
| 53 | 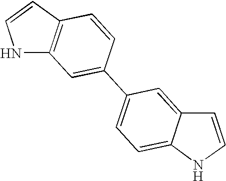 | 233 [M + H]^{+231 [M - H]}⁺ | δ (CDCl₃) = 6.57 (t, 1H), 6.60 (t, 1H), 7.19-7.23 (m, 2H), 7.42-7.53 (m, 3H), 7.62 (d, 1H), 7.69 (d, 1H), 7.89 (d, 1H), 8.12 (br. s, 2H) | ++ |
| 54 | 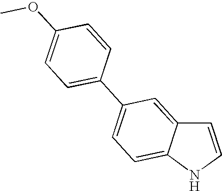 | 224 [M + H]^{+222 [M - H]}⁺ | δ (CDCl₃) = 3.83 (s, 3H), 6.96 (t, 1H), 6.99 (d, 2H), 7.17 (d, 1H), 7.38 (d, 2H), 7.55 (d, 2H), 7.79 (s, 1H), 8.06 (br. s, 1H) | ++ |
| 55 | 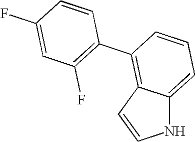 | 230 [M + H]^{+228 [M - H]}⁺ | | + |
| 56 | 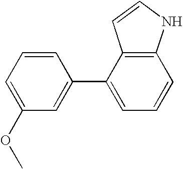 | 224 [M + H]^{+222 [M - H]}⁺ | δ (CDCl₃) = 3.88 (s, 3H0, 6.75 (dt, 1H), 6.91 (dd, 1H), 7.22 (dd, 1H), 7.27-7.31 (m, 4H), 7.39 (t, 2H), 8.26 (br. s, 1H) | + |
| 57 | 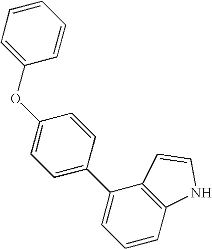 | 286 [M + H]^{+284 [M - H]}⁺ | δ (CDCl₃) = 6.65 (dt, 1H), 7.00-7.32 (m, 11H), 7.59 (dt, 2H), 8.16 (br. s, 1H) | + |
| 58 | 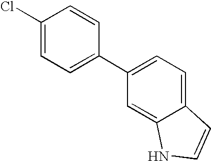 | 228 [M + H]^{+226 [M - H]}⁺ | δ (CDCl₃) = 6.50 (dt, 1H), 7.16 (s, 1H), 7.25 (dd, 1H), 7.31-7.34 (m, 2H), 7.47-7.51 (m, 3H), 7.62 (d, 1H), 8.13 (br. s, 1H) | + |
| 59 | 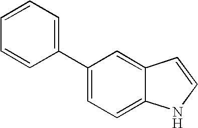 | 194 [M + H]^{+192 [M - H]}⁺ | δ (CDCl₃) = 6.51 (s, 1H), 7.09-7.60 (m, 8H), 7.77 (s, 1H), 8.00 (br. s, 1H) | + |
| 60 | 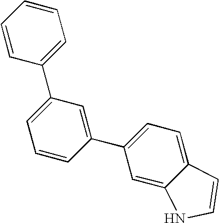 | 270 [M + H]^{+268 [M - H]}⁺ | δ (CDCl₃) = 6.53 (dt, 1H), 7.28-7.67 (m, 12H), 7.81 (t, 1H), 8.14 (br. s, 1H) | + |
| 61 | 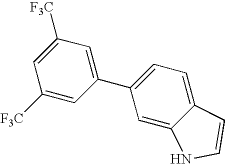 | 328 [M + H]^{+330 [M - H]}⁺ | δ (CDCl₃) = 6.55 (dt, 1H), 7.24 (t, 1H), 7.30 (dd, 1H), 7.57 (s, 1H), 7.68 (d, 1H), 7.74 (s, 1H), 8.00 (s, 2H), 8.22 (br. s, 1H) | + |
| 62 | 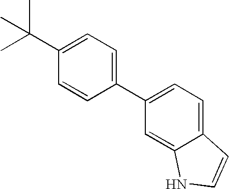 | 250 [M + H]^{+248 [M - H]}⁺ | δ (CDCl₃) = 1.37 (s, 9H), 6.57 (dt, 1H), 7.22 (t, 1H), 7.38 (dd, 1H), 7.45-7.48 (m, 2H), 7.58-7.60 (m, 3H), 7.67 (d, 1H), 8.18 (br. s, 1H) | + |

Biological Activity

The large conductance, voltage dependent and Ca²⁺-activated potassium channel BK is a potassium selective ion channel and belongs to the subfamily of K_{Ca} channels. Four BK alpha-subunits form a functional channel that can be regulated by intracellular Ca²⁺ concentration, membrane voltage, and other mechanisms like phosphorylation states or beta subunits. To test the biological activity of the compounds, we applied two different techniques, a fluorescence based assay using a voltage sensitive dye (Eₘ-Assay) as well as exploiting electrophysiological methods.

Eₘ-Assay:

CHO cells permanently transfected with cloned hSlo (α-hSlo and β-bSlo), yielding typical BK potassium currents (Zhou et al., Pflügers Arch., 436: 725-734 (1998), were used for the evaluation of compound activity. Activation or inhibition of BK channels in these cells leads to a change of the electrochemical gradient causing a hyperpolarized or depolarised membrane potential, respectively.

To determine changes in the membrane potential of the cells we used the voltage sensitive dye DiBAC₍₄₎3 (Molecular Probes) in a kinetic assay system using a fluorescent plate reader (Manning and Sontheimer, J. Neurosci. Meth., 91: 73-81 (1999). The anionic bis-oxonol DiBAC₍₄₎3 is a voltage sensitive dye which partitions from the extracellular environment into the cell where it reversibly binds to intracellular proteins, a kinetic process depending on the membrane potential of the cell. At depolarised potentials (i.e. at a reduced K⁺ efflux due to blocked K⁺ channels) the dye accumulates in the cell leading to an increased fluorescence intensity, due to its increased fluorescence if bound to cellular proteins. At hyperpolarized potentials (i.e. at an increased K⁺ efflux due to the opening of K⁺ channels), the dye partitions out of the cell causing a decreased fluorescence intensity.

hSlo transfected CHO cells where maintained in DMEM supplemented with 10% FCS, 250 μg/ml Geneticin, 100 μg/ml Hygromycin, 1×HT-Supplement, and 1×Non-essential Amino Acids and cultured in a humidified CO₂ incubator. After trypsination, cells where plated with a density of 5×10⁴ cells per well on a clear 96-well plate and incubated for 24 h. Cells where washed once with PBS, once with PBS containing 20 mM HEPES (adjusted to pH 7.4 with NaOH) and 2 μM DiBAC₍₄₎3 (DPBS-DiMAC solution). 180 μl of the dPBS-DiBAC solution was then added to the cells and the plate incubated for 30-60 min at 37° C. During this time the dye could partition into the cells and reach a certain steady-state distribution, depending on the resting membrane potential. Test and reference compounds were stored as DMSO stock solutions and diluted in dPBS-DiBAC solution to the desired concentration.

Fluorescence intensity (Ex.: 485 nm/Em: 520 nm) of each well was detected in the plate reader (Fluostar, BMG) every 60 seconds. After recording the baseline fluorescence for 7 minutes, 20 μl test and reference compounds were added and the fluorescence intensity was detected for additional 15 minutes. Background was subtracted, data values were normalized and expressed as a change in fluorescence intensity against time. The change in fluorescence intensity caused by the test compounds was evaluated compared to the effect of the reference compound NS004, and the ratio was determined (see Table I).

Electrophysiological Studies:

CHO cells permanently transfected with cloned α-hSlo and β-bSlo were maintained as described above and used for electrophysiologial characterisation. The whole-cell configuration of the patch-clamp technique was used to determine the effect of modulators on BK currents in these cells. The cell line expressing functional BK currents (Zhou et al., Pflügers Arch 436, p.725 (1998)) were plated onto glass cover slips with a density of 1-5×10⁴ cells/cover slip, incubated (37° C., 5% CO) and used for patch-clamp experiments within 24-48 h. Cells were bathed in mammalian ringer solutions containing (in mM): 160 NaCl, 4.5 KCl, 2CaCl₂, 1 MgCl₂, 10 HEPES, adjusted to pH 7.4, 290-310 mOsim. The internal pipette solution contained (in mM): 160 KCl, 2CaCl₂, 1 MgCl₂, 10 HEPES, EGTA was added to reach a free [Ca²⁺]ᵢₙₜₑᵣₙₐₗ=1×10 ⁻⁶M, adjusted to pH 7.2, 290-310 mOsm.

Borosilicate pipettes with a resistance of 2-3 MΩ were filled with the internal solution and mounted on an appropriate holder. Prior to measurements a recording chamber was mounted onto the cell-plated cover slips and the cells were perfused with a simple syringe driven perfusion system. Compounds were added in the final concentration (2×10⁵M) to the bath solution using the same system. An EPC-9 patch-clamp amplifier with Pulse and PulseFit software (HEKA) was used to record and analyze currents.

After addition of the compounds to the bath solution their modulating effect was determined by the increase or decrease of specific BK currents after reaching steady-state relative to the BK current before application of drugs (see Table II).

Table II: Results from the electrophysiological studies are given as the ratio of current increase after application of compound (20 μM) relative to the control current before compound application. Currents were determined after reaching steady-sate. Ranges are 1-1.1=+, >1,1-1.2=++, >1.2=+++

| Compound # | Mass | Effect |
|---|---|---|
| 1 | 277 | ++ |
| 2 | 236 | + |
| 3 | 229 | +++ |
| 4 | 261 | ++ |
| 5 | 223 | ++ |
| 22 | 233 | ++ |
| 30 | 227 | + |

1. The use of a compound of the Formula (I) 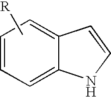
or a salt, a physiologically functional derivative, or a prodrug thereof as a medicament, wherein
R is a monocyclic or polycyclic substituted or unsubstituted aromatic ring system which may contain one or more groups X and which contains at least one aromatic ring;
X is selected from the group consisting of S, O, N, NR′, SO or SO₂;
R is optionally substituted by one to four substituents which are indepedently selected from the group consisting of halogen, CF₃, OCF₃, alkyl, cycloalkyl, haloalkyl, haloalkyloxy, hydroxyalkyl, hydroxyalkylamine, amine, aminoalkyl, alkylamine, CR′O, CO₂R′, alkoxy, alkylthio, arylalkyl, alkylsulfonyl, H, hydroxy, aryl, heteroaryl, —NR′OR′, CN, alkylsulfinyl, arylsulfonyl, heteroarylsulfonyl, SO₃R′, NO₂, —CO—NR′R¹, arylalkyl-O—, —O-aryl, —O-heteroaryl, arylalkyl-S—, —S-aryl, —S-heteroaryl, —NR¹—SO₂R′, —SO₂—NR¹-alkyl, —SO₂—NR¹-aryl, and —SO₂—NR¹-heteroaryl;
R′ is hydrogen, allyl, cycloalkyl, hydroxyalkyl, haloalkyl, hydroxyalkylamine, amine, alkylamine, arylalkyl, aryl or heteroaryl;
R¹ is hydrogen, hydroxy, alkyl, cycloalkyl, hydroxyalkyl, haloalkyl, arylalkyl, aryl or heteroaryl;
R² is hydrogen hydroxy, alkyl, cycloalkyl, hydroxyalkyl, haloalkyl, arylalkyl, aryl or heteroaryl, alkoxy;
wherein an alkyl group and the alkyl parts of the above groups denote a linear or branched chain of 1 to 6 carbon atoms which may contain one or more double bonds or one or more triple bonds and which is optionally substituted by one or more substituents R′, wherein R′ being as defined above;
an alkylsulfonyl group denotes an (SO)-alkyl group, the alkyl group being as defined above;
an alkylsulfinyl group denotes an (SO)-alkyl group, the alkyl group being as defined above;
a cycloalkyl group denotes a non-armoatic ring system, saturated or partially saturated, monocyclic or bicyclic carbocyclic alkyl containing 4 to 8 carbon atoms, wherein one or more of the carbon atoms in the ring can be substituted by a group X, X being as defined above. The cycloalkyl group is optionally substituted by one or more substituents R′, wherein R′ being as defined above;
an alkoxy group denotes an O-alkyl group, the alkyl group being as defined above;
an alkylthio group denotes an S-alkyl group, the alkyl group being as defined above;
a haloalkyl group denotes an alkyl group which is substituted by one to five halogen atoms, the alkyl group being as defined above;
a hydroxyalkyl group denotes an HO-alkyl group, the alkyl group being as defined above;
a haloalkyloxy group denotes an alkoxy group which is substituted by one to five halogen atoms, the alkyl group being as defined above;
a hydroxyalkylamine group denotes an (HO-alkyl)₂—N— group or HO-alkyl-NH— group, the alkyl group being as defined above;
an amine group denotes an NR¹R² group, R¹ and R² being as defined above;
an alkylamine group denotes an —NH-alkyl or —N-dialkyl group, the alkyl group being as defined above;
an aminoalkyl group denotes an H₂N-alkyl, monoalkylaminoalkyl, or dialkylaminoalkyl group, the alkyl group being as defined above.
a halogen group is chlorine, bromine, fluorine or iodine;
an aryl group denotes an aromatic group having 5 to 15 carbon atoms which is optionally substituted by one or more substituents R′, wherein R′ being as defined above;
an arylalkyl group denotes an alkyl group which is substituted by one to three preferably one aryl groups, the alkyl and aryl group being as defined above;
an arylsulfonyl group denotes an (SO₂)-aryl group, the aryl group being as defined above;
a heteroaryl group denotes a 5- or 6-membered heterocyclic group which contains at least one heteroatom O, N, or S, which is optionally fused to another ring and which is optionally substituted by one or more substituents R′, wherein R′ being as defined above;
a heteroarylsulfonyl group denotes an (SO₂)-heteroaryl group, the heteroaryl group being as defined above.

2. The use according to claim 1 wherein the medicament is used for the modulation of potassium channels.

3. The use according to claim 2 wherein the medicament is used for the prevention, alleviation or treatment of diseases, conditions or disorders which are associated with, or dependent on the membrane potential or conductance of cells in mammals, including a human.

4. The use according to claim 2 or 3 wherein the diseases are asthma, cystic fibrosis, obstructive pulmonary disease, convulsions, vascular spasms, urinary incontinence, urinary instability, urinary urgency, bladder spasms, ischemia cerebral ischemia, traumatic brain injury, neurodegeneration, migraine, pain, psychosis, hypertension, epilepsy, memory and attention deficits, functional bowel disorders, erectile dysfunction, immune suppression, autoimmune disorders, dysfunction of cellular proliferation, diabetes, premature labour, depression, shizophrenia, sleep disorders, other forms of headache, antipsychotic, or other disorders associated with or responsive to the modulation of potassium channels.
